SEQUENCE LISTING (3 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKANVDVLTKAKSQ

SEQ ID NO: 2 - protein, 34 aa, Homo sapiens
SVSEIQLMHNLGKHLNSMERVEWLRKKLQDVHNF

SEQ ID NO: 3 - protein, 31 aa, Homo sapiens
SVSEIQLMHNLGKHLNSMERVEWLRKKLQDV

(19) United States
(12) Patent Application Publication
(10) Pub. No.: US 2006/0189533 A1
(43) Pub. Date: Aug. 24, 2006

(54) Stable pharmaceutical dosage forms of teriparatide

(75) Inventors: Steven C. Quay, Seattle, WA (US); Henry R. Costantino, Woodinville, WA (US); Ching-Yuan Li, Bellevue, WA (US)

Correspondence Address: NASTECH PHARMACEUTICAL COMPANY INC, 3450 MONTE VILLA PARKWAY, BOTHELL, WA, 98021-8906, US

(73) Assignee: Nastech Pharmaceutical Company Inc.

(21) Appl. No.: 11/390,940

(22) Filed: Mar. 27, 2006

Related U.S. Application Data
(63) Continuation-in-part of application No. 11/347,554, filed on Feb. 3, 2006.
(63) Continuation-in-part of application No. 11/246,406, filed on Oct. 6, 2005.
(63) Continuation-in-part of application No. 11/246,450, filed on Oct. 6, 2005.
Application No. 11/246,450 is a continuation-in-part of application No. 11/126,996, filed on May 10, 2005.
(60) Provisional application No. 60/570,113, filed on May 10, 2004.

(51) Int. Cl. A61K 38/29

(52) U.S. Cl. 514/12

(57) ABSTRACT

A parathyroid hormone (1-34) (PTH) dosage form is described that is suitable for multi-use administration. A dosage form of parathyroid hormone (1-34) (PTH) comprising an aqueous pharmaceutical formulation for aerosolized intranasal delivery of PTH having a bioavailability of about 5% or greater, wherein the formulation comprises a therapeutically effective amount of PTH and polysorbate, and wherein least 90% of the PTH can be recovered after storage for 24 weeks at 5° C.

This application is a continuation-in-part and claims priority under 35 U.S.C. §120 of copending U.S. application Ser. No. 11/347,554 filed Feb. 3, 2006, Ser. No. 11/246,406 and Ser. No. 11/246,450 filed Oct. 6, 2005, which are continuation-in-part applications of copending U.S. application Ser. No. 11/126,996 filed May 10, 2005, and claims the benefit under 35 U.S.C. §119(e) of U.S. Provisional Application No. 60/570,113, filed May 10, 2004. All of the above applications are hereby incorporated by reference in their entirety.

### BACKGROUND OF THE INVENTION

The teachings of all the references cited in the present specification are incorporated in their entirety by reference.

Osteoporosis can be defined as a systemic skeletal disease characterized by low bone mass, microarchitectural deterioration of bone tissue, and increased bone fragility and susceptibility to fracture. It most commonly affects older populations, primarily postmenopausal women.

The prevalence of osteoporosis poses a serious health problem. The National Osteoporosis Foundation has estimated that 44 million people are experiencing the effects of osteoporosis or osteopenia. By the year 2010, osteoporosis will affect more than 52 million people and, by 2020, more than 61 million people. The prevalence of osteoporosis is greater in Caucasians and Asians than in African-Americans, perhaps because African-Americans have a higher peak bone mass. Women are affected in greater numbers than men because men have a higher peak bone density. Furthermore, as women age the rate of bone turnover increases, resulting in accelerated bone loss because of the lack of estrogen after menopause.

The goal of pharmacological treatment of osteoporosis is to maintain or increase bone strength, to prevent fractures throughout the patient's life, and to minimize osteoporosis-related morbidity and mortality by safely reducing the risk of fracture. The medications that have been used most commonly to treat osteoporosis include calcium, and vitamin D, estrogen (with or without progestin), bisphonates, selective estrogen receptor modulators (SERMs), and calcitonin.

Parathyroid hormone (PTH) has recently emerged as a popular osteoporosis treatment. Unlike other therapies that reduce bone resorption, PTH increases bone mass, which results in greater bone mineral density (BMD). PTH has multiple actions on bone, some direct and some indirect. PTH increases the rate of calcium release from bone into blood. The chronic effects of PTH are to increase the number of bone cells both osteoblasts and osteoclasts, and to increase the remodeling bone. These effects are apparent within hours after PTH is administered and persist for hours after PTH is withdrawn. PTH administered to osteoporotic patients leads to a net stimulation of bone formation especially in trabecular bone in the spine and hip resulting in a highly significant reduction in fractures. The bone formation is believed to occur by the stimulation of osteoblasts by PTH as osteoblasts have PTH receptors.

Parathyroid hormone (PTH) is a secreted, 84 amino acid residue polypeptide having the amino acid sequence Ser-Val-Ser-Glu-Ile-Gln-Leu-Met-His-Asn-Leu-Gly-Lys -His-Leu-Asn-Ser-Met-Glu-Arg-Val-Glu-Trp-Leu-Arg-Lys-Lys-Leu-Gln-Asp-Val-His-Asn-Phe Val Ala Leu Gly Ala Pro Leu Ala Pro Arg Asp Ala Gly Ser Gln Arg Pro Arg Lys Lys Glu Asp Asn Val Leu Val Glu Ser His Glu Lys Ser Leu Gly Glu Ala Asp Lys Ala Asn Val Asp Val Leu Thr Lys Ala Lys Ser Gln (SEQ ID NO: 1). Studies in humans with certain forms of PTH have demonstrated an anabolic effect on bone, and have prompted significant interest in its use for the treatment of osteoporosis and related bone disorders.

Using the N-terminal 34 amino acids of the bovine and human hormone Ser-Val-Ser -Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-His-Asn-Phe (SEQ ID NO: 2) for example, which by all published accounts are deemed biologically equivalent to the full length hormone, it has been demonstrated in humans that parathyroid hormone enhances bone growth particularly when administered in pulsatile fashion by the subcutaneous route. A slightly different form of PTH, human PTH (1-38) has shown similar results.

PTH (1-34), also called teriparatide, is currently on the market under the brand name FORTEO®, Eli Lilly, Indianapolis, Ind. for the treatment of postmenopausal women with osteoporosis who are at high risk of fracture. This drug is administered by a once daily subcutaneous injection of 20 μg in a solution containing acetate buffer, mannitol, and m-cresol in water, pH 4. However, many people are adverse to injections, and thus become non-compliant with the prescribed dosing of the PTH. Thus, there is a need to develop an intranasal formulation of a parathyroid hormone peptide that has suitable bioavailability such that therapeutic levels can be achieved in the blood to be effective to treat osteoporosis or osteopenia. FORTEO® is manufactured by recombinant DNA technology using an Escherichia coli strain. PTH (1 -34) has a molecular weight of 4117.87 daltons. Reviews on PTH (1-34) and its clinical uses have been published, including, e.g., Brixen et al, 2004; Dobnig, 2004; Eriksen and Robins, 2004; Quattrocchi and Kourlas 2004, are hereby incorporated by reference. FORSTEO is currently licensed in the US (as FORTEO®) and Europe. The safety of teriparatide has been evaluated in over 2800 patients in doses ranging from 5 to 100 μg per day in short term trials. Doses of up to 40 μg per day have been given for up to two years in long term trials. Adverse events associated with FORSTEO were usually mild and generally did not require discontinuation of therapy. The most commonly reported adverse effects were dizziness, leg cramps, nausea, vomiting and headache. Mild transient hypercalcemia has been reported with FORSTEO which is usually self limiting within 6 hours.

Currently FORTEO® is administered as a daily subcutaneous injection. The following Cmax and AUC values are described for various doses of FORTEO (20 ug is the commercially approved dose).

| SC Dose | | CL/F | AUC₀₋ₜ | Cₘₐₓ |
|---|---|---|---|---|
| (μg) | N | (L/hr) | (pg hr/ml) | (pg/ml) |
| 20 | 22 | 152.3 ± 91.2 | 165 ± 67.6 | 151.0 ± 56.9 |
| 40 | 16 | 124.3 ± 65.8 | 393 ± 161 | 256.2 ± 117.5 |
| 80 | 22 | 104.4 ± 27.9 | 816 ± 202.2 | 552.8 ± 183.6 |

It would be preferable for patient acceptability if a non-injected route of administration were available, including nasal, bucal, gastrointestinal and dermal. Teriparatide has previously been administered intranasally to humans at doses of up to 500 μg per day for 7 days in one study (Suntory News Release). Suntory Establishes Large Scale Production of recombinant human PTH₁₋₃₄ and obtains promising results from Phase 1 Clinical Trials using a Nasal Formulation. February 1999. http://www.suntory.com/news/1999-02.html accessed 15 Apr. 2004) and in another study subjects received up to 1,000 μg per day for 3 months (Matsumoto et al. Daily Nasal Spray of hPTH₁₋₃₄ for 3 Months Increases Bone Mass in Osteoporotic Subjects (ASBMR 2004 presentation 1171 October 4, 2004, Seattle Wash.), no safety concerns were noted with this route.

Most PTH formulations are reconstituted from fresh or lyophilized hormone, and incorporate various carriers, excipients and vehicles. PTH formulations are often prepared in water-based vehicles such as saline, or water which is acidified typically with acetic acid to solubilize the hormone. Many reported formulations also incorporate albumin as a stabilizer (see for example Reeve at al., Br. Med. J., 1980, 280:6228; Reeve at al., Lancet, 1976, 1:1035; Reeve at al., Calcif. Tissue Res., 1976, 21:469; Hodsman et al., Bone Miner 1990, 9(2):137; Tsai et al., J. Clin. Endocrinol Metab., 1989, 69(5):1024; Isaac et al., Horm. Metab. Res., 1980, 12(9):487; Law et al., J. Clin Invest. 1983, 72(3):1106; and Hulter, J. Clin Hypertens, 1986, 2(4):360). Other reported formulations incorporate an excipient such as mannitol with either lyophilized hormone or in the reconstituted vehicle. Some formulations used for human studies include a human PTH (1-34) preparation consisting of mannitol, heat inactivated human serum albumin, and caproic acid (a protease inhibitor) as an absorption enhancer (see Reeve at al., 1976, Calcif. Tissue Res., 21, Suppl., 469-477); a human PTH (1-38) preparation reconstituted into a saline vehicle (see Hodsman et al., 1991, 14(1), 67-83); and a bovine PTH (1-34) preparation in aqueous vehicle pH adjusted with acetic acid and containing albumin. The International Reference preparation for human PTH (1-84) consists of 100 ng of hormone ampouled with 250 .mu.g human serum albumin and 1.25 mg lactose (1981), and for bovine PTH (1-84) consists of 10 .mu.g lyophilized hormone in 0.01 M acetic acid and 0.1% w/v mannitol (see Martindale, The Extra Pharmacoepia, The Pharmaceutical Press. London, 29th Edition, 1989 at p. 1338). A formulation aimed at improving the stability for a lyophilized preparation of h-PTH (1-34) is reported in EP 619 119 using a combination of sugar and sodium chloride. U.S. Pat. No. 5,496,801 describes a freeze-dried composition for the natural hormone, PTH (1-84), containing mannitol as an excipient and a citrate source as a non-volatile buffering agent.

U.S. Pat. No. 6,770,623 describes stabilized teriparatide formulations. The '623 formulations require a buffer. The buffering agent includes any acid or salt combination which is pharmaceutically acceptable and capable of maintaining the aqueous solution at a pH range of 3 to 7, preferably 3-6, e.g., acetate, tartrate or citrate sources. The concentration of buffer may be in the range of about 2 mM to about 500 mM.

U.S. Pat. No. 5,407,911 describes the use of dipotassium glycyrrhizate as an emulsifying agent for nasal administration of PTH. Polysorbate 80 was determined to be inferior when used in the intranasal PTH formulations because it caused a precipitate and instability in the formulation.

Commercial exploitation of parathyroid hormone requires the development of a formulation that is acceptable in terms of storage stability and ease of preparation. Because it is a protein and thus far more labile than traditional small molecular weight drugs, a parathyroid hormone formulation presents challenges not commonly encountered by the pharmaceutical industry. Furthermore, like other proteins that have been formulated successfully, PTH is particularly sensitive to oxidation, deamidation and hydrolysis, and requires that its N-terminal and C-terminal sequences remain intact in order to preserve bioactivity.

Formulating proteins is generally more difficult that formulating small molecules, because proteins are more susceptible to degradation (see Arakawa et al. (2001) Adv. Drug Del. Rev. 46:307-26, hereby incorporated by reference in its entirety). Thus, the stability of purified proteins is difficult to predict a priori and in general must be assessed on a case-by-case basis. Forteo is a liquid pharmaceutical formulation of teriparatide that requires a buffer for its stability. There remains a need for a storage-stable formulation of teriparatide that does not require a buffer, and is suitable for intranasal administration.

### SUMMARY OF THE INVENTION

One aspect of the invention is a dosage form of parathyroid hormone (1-34) (PTH) comprising an aqueous pharmaceutical formulation for aerosolized intranasal delivery of PTH having a bioavailability of about 5% or greater, wherein the formulation comprises a therapeutically effective amount of PTH and polysorbate, and wherein least 90% of the PTH can be recovered after storage for 24 weeks at 5° C. In one embodiment, the PTH dosage form has greater than about 90% recovery of the PTH after at least six months at 5° C. storage.

In another embodiment, the PTH dosage form has greater than about 90% recovery of the PTH after one year at 5° C. storage. In another embodiment, the PTH dosage form has greater than about 90% recovery of the PTH after two years at 5° C. storage. In another embodiment, the PTH dosage form has greater than about 80% recovery of the PTH after 24 weeks at 25° C. storage. In another embodiment, the PTH dosage form has greater than about 80% recovery of the PTH after at least six months at 25° C. storage. In another embodiment, the PTH dosage form has greater than about 80% recovery of the PTH after one year at 25° C. storage. In another embodiment, the PTH dosage form has greater than about 80% recovery of the PTH after two years at 25° C. storage. In another embodiment, the PTH dosage form has greater than about 65% recovery of the PTH can be recovered after storage for at least 4 weeks at 40° C. In another embodiment, the PTH dosage form has greater than about 90% recovery of the PTH after being in use for greater than about five days. In another embodiment, the PTH dosage form has greater than about 90% recovery of PTH at 30° C./65% relative humidity between all sprays. In another embodiment, the pH is about 5.0 or less. In another embodiment, the pH is about 4.5 or less. In another embodiment, the pH is about 4.0 or less. In another embodiment, the pH is about 3.5 or less. In another embodiment, the concentration of PTHis at least about 1 mg/ml or greater. In another embodiment, the concentration of PTH is at least about 2 mg/ml or greater. In another embodiment, concentration of PTH is at least about 6 mg/ml or greater. In another embodiment, the concentration of PTH is at least about 10 mg/ml or greater. In another embodiment, the PTH dosage form is suitable for intra-nasal administration to achieve a dose of from about 2 μg to about 1000 μg of said PTH. In another embodiment, the PTH dosage form is suitable for intra-nasal administration to achieve a dose of from about 100 μg to about 600 μg of said PTH. In another embodiment, polysorbate is present at least about 1 mg/mL in the formulation. In another embodiment, polysorbate is present at least about 10 mg/mL in the formulation. In another embodiment, polysorbate is present at least about 50 mg/mL in the formulation. In anothere embodiment, the PTH dosage is further comprising a preservative. In another embodiment, the preservative is chlorobutanol.

Another aspect of the invention is a dosage form of parathyroid hormone (1-34) (PTH) comprising an aqueous pharmaceutical formulation for aerosolized intranasal delivery of PTH having a bioavailability of about 10% or greater, wherein the formulation comprises a therapeutically effective amount of PTH, methyl-β-cyclodextrin, didecanoylphosphatidyl choline, and ethylenediaminetetraacetic acid, and wherein least 90% of the PTH can be recovered after storage for 24 weeks at 5° C. In one embodiment, the PTH dosage form has greater than about 90% recovery of PTH after at least six months at 5° C. storage. In another embodiment, the PTH dosage form has greater than about 90% recovery of PTH after one year at 5° C. storage. In another embodiment, the PTH dosage form has greater than about 90% recovery of PTH after two years at 5° C. storage. In another embodiment, the PTH dosage form has greater than about 80% recovery of PTH after 24 weeks at 25° C. storage. In another embodiment, the PTH dosage form has greater than about 80% recovery of PTH after at least six months at 25° C. storage. In another embodiment, the PTH dosage form has greater than about 80% recovery of PTH after one year at 25° C. storage. In another embodiment, the PTH dosage form has greater than about 80% recovery of PTH after two years at 25° C. storage. In another embodiment, the PTH dosage form has greater than about 65% recovery of the PTH after storage for at least 4 weeks at 40° C. In another embodiment, the PTH dosage form has greater than about 90% recovery of the PTH after being in use for greater than about five days. In another embodiment, the PTH dosage form has greater than about 90% recovery of PTH at 30° C./65% relative humidity between all sprays. In another embodiment, the pH is about 5.0 or less. In another embodiment, the pH is about 4.5 or less. In another embodiment, the pH is about 4.0 or less. In another embodiment, the pH is about 3.5 or less. In another embodiment, the concentration of PTH is at least about 1 mg/ml or greater. In another embodiment, the concentration of PTH is at least about 2 mg/ml or greater. In another embodiment, the concentration of PTH is at least about 6 mg/ml or greater. In another embodiment, the concentration of PTH is at least about 10 mg/ml or greater. In another embodiment, the PTH dosage form is suitable for intra-nasal administration to achieve a dose of from about 2 μg to about 1000 μg of said PTH. In another embodiment, the PTH dosage form is suitable for intra-nasal administration to achieve a dose of from about 100 μg to about 600 μg of said PTH. In another embodiment, the PTH dosage form is futher comprising a preservative. In another embodiment, the preservative is chlorobutanol.

### BRIEF DISCRIPTION OF THE DRAWINGS

FIG. 1: Mean Plasma Concentration versus Time for Periods 1-5: (Linear Graph).

FIG. 2: Ratio of Cₘₐₓ to Mean, Low Dose PTH Formulations versus Forsteo.

### DISCLOSURE OF THE INVENTION

Preferably the hormone is parathyroid hormone and the mammal is a human. In a most preferred embodiment the parathyroid hormone peptide is PTH (1-34), also known as teriparatide. Tregear, U.S. Pat. No. 4,086,196, described human PTH analogues and claimed that the first 27 to 34 amino acids are the most effective in terms of the stimulation of adenylyl cyclase in an in vitro cell assay. Pang et al, WO93/06845, published Apr. 15, 1993, described analogues of human PTH which involve substitutions of Arg²⁵, Lys²⁶, Lys²⁷ with numerous amino acids, including alanine, asparagine, aspartic acid, cysteine, glutamine, glutamic acid, glycine, histidine, isoleucine, leucine, methionine, phenylalanine, proline, serine, threonine, tryptophan, tyrosine, or valine. Other PTH analogues are disclosed in the following patents, hereby incorporated by reference: U.S. Pat. No. 5,317,010; U.S. Pat. No. 4,822,609; U.S. Pat. No. 5,693.616; U.S. Pat. No. 5,589,452; U.S. Pat. No. 4,833,125; U.S. Pat. No. 5,607,915; U.S. Pat. No. 5,556,940; U.S. Pat. No. 5,382,658; U.S. Pat. No. 5,407,911 ;U.S. Pat. No. 6,583,114; U.S. Pat. No. 6,541,450; U.S. Pat. No. 6,376,502; U.S. Pat. No. 5,955,425; U.S. Pat. No. 6,316,410; U.S. Pat. No. 6,110,892; U.S. Pat. No. 6,051,686; U.S. Pat. No. 5,695,955; U.S. Pat. No. 4,771,124; and U.S. Pat. No. 6,376,502.

PTH operates through activation of two second messenger systems, Gₛ-protein activated adenylyl cyclase (AC) and G_{q}-protein activated phospholipase C_{β}. The latter system results in a stimulation of membrane-bound protein kinase C (PKC) activity. The PKC activity has been shown to require PTH residues 29 to 32 (Jouishomme et al (1994) J. Bone Mineral Res. 9, (1179-1189). It has been established that the increase in bone growth, i.e. the effect which is useful in the treatment of osteoporosis, is coupled to the ability of the peptide sequence to increase AC activity. The native PTH sequence, PTH (1-84) (SEQ ID NO: 1), has been shown to have all of these activities.

The following linear analogue, hPTH₁₋₃₁NH₂, has only AC-stimulating activity and has been shown to be fully active in the restoration of bone loss in the ovariectomized rat model [Rixon, R. H. et al., J Bone Miner. Res. 9: 1179-1189 (1994)]; Whitfield et al., Calcified Tissue Int. 58: 81-87 (1996)]; Willick et al, U.S. Pat. No. 5,556,940, hereby incorporated by reference: Ser Val Ser Glu Ile Gln Leu Met His Asn Leu Gly Lys His Leu Asn Ser Met Glu Arg Val Glu Trp Leu Arg Lys Lys Leu Gln Asp Val (SEQ ID NO: 3). The above molecule, SEQ ID NO:3, and its counterpart with a Leu₂₇ substitution SEQ ID NO:2 may have a free carboxyl ending instead of the amide ending. Another PTH analog is [Leu₂₇]cyclo(Glu₂₂-Lys₂₆)PTH₁₋₃₁.

Parathyroid hormone fragments desirably incorporate at least the first 34 N-terminal residues, such as PTH (1-34) (SEQ ID NO: 2), PTH (1-37), PTH (1-38) and PTH (1-41). Alternatives in the form of PTH variants incorporate from 1 to 5 amino acid substitutions that improve PTH stability and half-life, such as the replacement of methionine residues at positions 8 and/or 18 with leucine or other hydrophobic amino acid that improves PTH stability against oxidation and the replacement of amino acids in the 25-27 region with trypsin-insensitive amino acids such as histidine or other amino acid that improves PTH stability against protease.

The above described forms of parathyroid hormone are embraced by the terms “parathyroid hormone” or “PTH” or “PTH peptide” as used generically herein. The parathyroid hormones may be obtained by known recombinant or synthetic methods, such as described in U.S. Pat. No. 4,086,196 incorporated herein by reference.

Thus, the present invention is a method for treating osteoporosis or osteopenia in a mammal comprising transmucosally administering a formulation comprised of a PTH peptide, such that when 50 μg of the PTH is administered transmucosally to the mammal the concentration of the PTH peptide in the plasma of the mammal increases by at least 5 pmol, preferably at least 10 pmol per liter of plasma.

Intranasal delivery-enhancing agents are employed which enhance delivery of PTH into or across a nasal mucosal surface. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The intranasal delivery-enhancing agent of the present invention may be a pH control agent. The pH of the pharmaceutical formulation of the present invention is a factor affecting absorption of PTH via paracellular and transcellular pathways to drug transport. In one embodiment, the pharmaceutical formulation of the present invention is pH adjusted to between about pH 3.0 to 7.0. In a further embodiment, the pharmaceutical formulation of the present invention is pH adjusted to between about pH 3.0 to 6.0. In a further embodiment, the pharmaceutical formulation of the present invention is pH adjusted to between about pH 4.0 to 5.0. Generally, the pH is 4.0±0.3.

As noted above, the present invention provides improved methods and compositions for mucosal delivery of PTH peptide to mammalian subjects for treatment or prevention of osteoporosis or osteopenia. Examples of appropriate mammalian subjects for treatment and prophylaxis according to the methods of the invention include, but are not restricted to, humans and non-human primates, livestock species, such as horses, cattle, sheep, and goats, and research and domestic species, including dogs, cats, mice, rats, guinea pigs, and rabbits.

In order to provide better understanding of the present invention, the following definitions are provided:

According to the present invention a PTH peptide also includes the free bases, acid addition salts or metal salts, such as potassium or sodium salts of the peptides, and PTH peptides that have been modified by such processes as arnidation, glycosylation, acylation, sulfation, phosphorylation, acetylation, cyclization and other well known covalent modification methods.

The nasal spray product manufacturing process generally includes the preparation of a diluent for PTH (1-34) nasal spray, which includes ˜85% water plus the components of the nasal spray formulation without PTH. The pH of the diluent is then measured and adjusted to pH 4.0±0.3 with sodium hydroxide or hydrochloric acid, if necessary. The PTH (1-34) nasal spray is prepared by the non-aseptic transfer of ˜85% of the final target volume of the diluent to a screw cap bottle. An appropriate amount of PTH (1-34) is added and mixed until completely dissolved. The pH is measured and adjusted to pH 4.0±0.3 with sodium hydroxide or hydrochloric acid, if necessary. A sufficient quantity of diluent is added to reach the final target volume. Screw-cap bottles are filled and caps affixed. The above description of the manufacturing process represents a method used to prepare the initial clinical batches of drug product. This method may be modified during the development process to optimize the pharmaceutical properties.

Currently marketed PTH requires sterile manufacturing conditions for compliance with FDA regulations. Parenteral administration, including PTH for injection or infusion, requires a sterile (aseptic) manufacturing process. Current Good Manufacturing Practices (GMP) for sterile drug manufacturing include standards for design and construction features (21 CFR §211.42 (Apr. 1, 2005)); standards for testing and approval or rejection of components, drug product containers, and closures (§211.84); standards for control of microbiological contamination (§211.113); and other special testing requirements (§211.167). Non-parenteral (non-aseptic) products, such as the intranasal product of the invention, do not require these specialized sterile manufacturing conditions. As can be readily appreciated, the requirements for a sterile manufacturing process are substantially higher and correspondingly more costly than those required for a non-sterile product manufacturing process. These costs include much greater capitalization costs for facilities, as well as a more costly manufacturing cost: extra facilites for sterile manufacturing include additional rooms and ventilation; extra costs associated with sterile manufacturing include greater manpower, extensive quality control and quality assurance, and administrative support. As a result, manufacturing costs of an intranasal PTH product, such as that of the invention, are far less than those of a parenterally administered PTH product. The present invention satisfies the need for a non-sterile manufacturing process for PTH.

“Mucosal delivery-enhancing agents” are defined as chemicals and other excipients that, when added to an aqueous PTH formulation results in a formulation that produces a significant increase in transport of PTH peptide across a mucosa as measured by the maximum blood, serum, or cerebral spinal fluid concentration (Cₘₐₓ) or by the area under the curve, AUC, in a plot of concentration versus time. A mucosa includes the nasal, oral, intestional, buccal, bronchopulmonary, vaginal, and rectal mucosal surfaces and in fact includes all mucus-secreting membranes lining all body cavities or passages that communicate with the exterior. Mucosal delivery enhancing agents are sometimes called carriers.

As used herein, mucosal delivery-enhancing agents include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of PTH peptide or other biologically active compound(s). Enhancement of mucosal delivery can thus occur by any of a variety of mechanisms, for example by increasing the diffusion, transport, persistence or stability of PTH peptide, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

As used herein, a “mucosally effective amount of PTH peptide” contemplates effective mucosal delivery of PTH peptide to a target site for drug activity in the subject that may involve a variety of delivery or transfer routes. For example, a given active agent may find its way through clearances between cells of the mucosa and reach an adjacent vascular wall, while by another route the agent may, either passively or actively, be taken up into mucosal cells to act within the cells or be discharged or transported out of the cells to reach a secondary target site, such as the systemic circulation. The methods and compositions of the invention may promote the translocation of active agents along one or more such alternate routes, or may act directly on the mucosal tissue or proximal vascular tissue to promote absorption or penetration of the active agent(s). The promotion of absorption or penetration in this context is not limited to these mechanisms.

As used herein “peak concentration (Cₘₐₓ) of PTH peptide in a blood plasma”, “area under concentration vs. time curve (AUC) of PTH peptide in a blood plasma”, “time to maximal plasma concentration (tₘₐₓ) of PTH peptide in a blood plasma” are pharmacokinetic parameters known to one skilled in the art. Laursen et al., Eur. J. Endocrinology, 135: 309-315 (1996). The “concentration vs. time curve” measures the concentration of PTH peptide in a blood serum of a subject vs. time after administration of a dosage of PTH peptide to the subject either by intranasal, intramuscular, or subcutaneous route of administration. “Cₘₐₓ” is the maximum concentration of PTH peptide in the blood serum of a subject following a single dosage of PTH peptide to the subject. “tₘₐₓ” is the time to reach maximum concentration of PTH peptide in a blood serum of a subject following administration of a single dosage of PTH peptide to the subject.

A “buffer” is generally used to maintain the pH of a solution at a nearly constant value. A buffer maintains the pH of a solution, even when small amounts of strong acid or strong base are added to the solution, by preventing or neutralizing large changes in concentrations of hydrogen and hydroxide ions. A buffer generally consists of a weak acid and its appropriate salt (or a weak base and its appropriate salt). The appropriate salt for a weak acid contains the same negative ion as present in the weak acid. (see Lagowski, Macmillan Encyclopedia of Chemistry, Vol. 1, Simon & Schuster, New York, 1997 at p. 273-4). The Henderson-Hasselbach Equation, pH=pKa +log₁₀[A⁻]/[HA], is used to describe a buffer, and is based on the standard equation for weak acid dissociation, HA [symbol] H⁺+A⁻. Examples of commonly used buffer sources include the following: acetate, tartrate or citrate.

The “buffer capacity” means the amount of acid or base that can be added to a buffer solution before a significant pH change will occur. If the pH lies within the range of pK−1 and pK+1 of the weak acid the buffer capacity is appreciable, but outside this range it falls off to such an extent as to be of little value. Therefore, a given system only has a useful buffer action in a range of one pH unit on either side of the pK of the weak acid (or weak base). (see Dawson, Data for Biochemical Research, Third Edition, Oxford Science Publications, 1986 at p. 419). Generally, suitable concentrations are chosen so that the pH of the solution is close to the pKa of the weak acid (or weak base). (see Lide, CRC Handbook of Chemistry and Physics, 86^{th} Edition, Taylor & Francis Group, 2005-2006 at chap.-p. 2-41). Further, solutions of strong acids and bases are not normally classified as buffer solutions, and they do not display buffer capacity between pH values 2.4 to 11.6.

“Non-infused administration” means any method of delivery that does not involve an injection directly into an artery or vein, a method which forces or drives (typically a fluid) into something and especially to introduce into a body part by means of a needle, syringe or other invasive method. Non-infused administration includes subcutaneous injection, intramuscular injection, intraparitoneal injection and the non-injection methods of delivery to a mucosa.

Osteoporosis is a systemic skeletal disease characterized by low bone mass, microarchitectural deterioration of bone tissue, and increased bone fragility and susceptibility to fracture. Osteopenia is a decreased calcification or density of bone, a descriptive term applicable to all skeletal systems in which the condition is noted.

Osteoporosis or osteopenia therapies and medical diagnosis include the administration of a clinically effective dose of PTH for the prevention and/or treatment of osteoporosis or osteopenia. As noted above, the instant invention provides improved and useful methods and compositions for nasal mucosal delivery of a PTH peptide to prevent and treat osteoporosis or osteopenia in mammalian subjects. As used herein, prevention and treatment of osteoporosis or osteopenia means prevention of the onset or lowering the incidence or severity of clinical osteoporosis by reducing increasing bone mass, decreasing bone resporption, or reducing the incidence of fractured bones in a patient.

The PTH peptide can also be administered in conjunction with other therapeutic agents such as bisphonates, calcium, vitamin D, estrogen or estrogen-receptor binding compounds, selective estrogen receptor modulators (SERMs), bone morphogenic proteins, or calcitonin.

Improved methods and compositions for mucosal administration of PTH peptide to mammalian subjects optimize PTH peptide dosing schedules. The present invention provides mucosal delivery of PTH peptide, formulated with one or more mucosal delivery enhancing agents, wherein PTH peptide dosage release is substantially normalized and/or sustained for an effective delivery period of PTH peptide release ranging from approximately 0.1 to 2.0 hours; 0.4 to 1.5 hours; 0.7 to 1.5 hours; or 0.8 to 1.0 hours; following mucosal administration. The sustained release of PTH peptide may be facilitated by repeated administration of exogenous PTH peptide utilizing methods and compositions of the present invention.

Improved compositions and methods for mucosal administration of PTH peptide to mammalian subjects optimize PTH peptide dosing schedules. The present invention provides improved mucosal (e.g., nasal) delivery of a formulation comprising PTH peptide in combination with one or more mucosal delivery-enhancing agents and an optional sustained release-enhancing agent or agents. Mucosal delivery-enhancing agents of the present invention yield an effective increase in delivery, e.g., an increase in the maximal plasma concentration (Cₘₐₓ) to enhance the therapeutic activity of mucosally-administered PTH peptide. A second factor affecting therapeutic activity of PTH peptide in the blood plasma and CNS is residence time (RT). Sustained release-enhancing agents, in combination with intranasal delivery-enhancing agents, increase Cₘₐₓ and increase residence time (RT) of PTH peptide. Polymeric delivery vehicles and other agents and methods of the present invention that yield sustained release-enhancing formulations, for example, polyethylene glycol (PEG), are disclosed herein. The present invention provides an improved PTH peptide delivery method and dosage form for treatment or prevention of osteoporosis or osteopenia in mammalian subjects.

Within the mucosal delivery compositions and methods of the invention, various delivery-enhancing agents are employed which enhance delivery of PTH peptide into or across a mucosal surface. In this regard, delivery of PTH peptide across the mucosal epithelium can occur “transcellularly” or “paracellularly.” The extent to which these pathways contribute to the overall flux and bioavailability of the PTH peptide depends upon the environment of the mucosa, the physico-chemical properties the active agent, and the properties of the mucosal epithelium. Paracellular transport involves only passive diffusion, whereas transcellular transport can occur by passive, facilitated, or active processes. Generally, hydrophilic, passively transported, polar solutes diffuse through the paracellular route, while more lipophilic solutes use the transcellular route. Absorption and bioavailability (e.g., as reflected by a permeability coefficient or physiological assay), for diverse, passively and actively absorbed solutes, can be readily evaluated, in terms of both paracellular and transcellular delivery components, for any selected PTH peptide within the invention. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area and the size- and charge-based discrimination against macromolecular permeation suggest that the paracellular route is a generally less favorable route than transcellular delivery for drug transport. Surprisingly, the methods and compositions of the invention provide for significantly enhanced transport of biotherapeutics into and across mucosal epithelia via the paracellular route. Therefore, the methods and compositions of the invention successfully target both paracellular and transcellular routes, alternatively, or within a single method or composition.

While the mechanism of absorption promotion may vary with different mucosal delivery-enhancing agents of the invention, useful reagents in this context will not substantially adversely affect the mucosal tissue and is selected according to the physicochemical characteristics of the particular PTH peptide or other active or delivery-enhancing agent. In this context, delivery-enhancing agents that increase penetration or permeability of mucosal tissues will often result in some alteration of the protective permeability barrier of the mucosa. For such delivery-enhancing agents to be of value within the invention, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of the invention, absorption-promoting agents for coordinate administration or combinatorial formulation with PTH peptide of the invention are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium laurylsulfate, oleic acid, and the bile salts, may be employed to enhance mucosal penetration of the PTH peptide. In additional aspects, surfactants (e.g., polysorbates) are employed as adjunct compounds, processing agents, or formulation additives to enhance intranasal delivery of the PTH peptide. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the PTH peptide from the vehicle into the mucosa.

Additional mucosal delivery-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other absorption-promoting agents are selected from a variety of carriers, bases and excipients that enhance mucosal delivery, stability, activity or trans-epithelial penetration of the PTH peptide. These include, inter alia, cyclodextrins and β-cyclodextrin derivatives (e.g., 2-hydroxypropyl-β-cyclodextrin and heptakis(2,6-di-O-methyl-β-cyclodextrin). These compounds, optionally conjugated with one or more of the active ingredients and further optionally formulated in an oleaginous base, enhance bioavailability in the mucosal formulations of the invention. Yet additional absorption-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

The mucosal therapeutic and prophylactic compositions of the present invention may be supplemented with any suitable penetration-promoting agent that facilitates absorption, diffusion, or penetration of PTH peptide across mucosal barriers. The penetration promoter may be any promoter that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Within various aspects of the invention, improved nasal mucosal delivery formulations and methods are provided that allow delivery of PTH peptide and other therapeutic agents within the invention across mucosal barriers between administration and selected target sites. Certain formulations are specifically adapted for a selected target cell, tissue or organ, or even a particular disease state. In other aspects, formulations and methods provide for efficient, selective endo- or transcytosis of PTH peptide specifically routed along a defined intracellular or intercellular pathway. Typically, the PTH peptide is efficiently loaded at effective concentration levels in a carrier or other delivery vehicle, and is delivered and maintained in a stabilized form, e.g., at the nasal mucosa and/or during passage through intracellular compartments and membranes to a remote target site for drug action (e.g., the blood stream or a defined tissue, organ, or extracellular compartment). The PTH peptide may be provided in a delivery vehicle or otherwise modified (e.g., in the form of a prodrug), wherein release or activation of the PTH peptide is triggered by a physiological stimulus (e.g. pH change, lysosomal enzymes, etc.). Often, the PTH peptide is pharmacologically inactive until it reaches its target site for activity. In most cases, the PTH peptide and other formulation components are non-toxic and non-immunogenic. In this context, carriers and other formulation components are generally selected for their ability to be rapidly degraded and excreted under physiological conditions. At the same time, formulations are chemically and physically stable in dosage form for effective storage.

Included within the definition of biologically active peptides and proteins for use within the invention are natural or synthetic, therapeutically or prophylactically active, peptides (comprised of two or more covalently linked amino acids), proteins, peptide or protein fragments, peptide or protein analogs, and chemically modified derivatives or salts of active peptides or proteins. A wide variety of useful analogs and mimetics of PTH peptide are contemplated for use within the invention and can be produced and tested for biological activity according to known methods. Often, the peptides or proteins of PTH peptide or other biologically active peptides or proteins for use within the invention are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine. By aligning a peptide or protein analog optimally with a corresponding native peptide or protein, and by using appropriate assays, e.g., adhesion protein or receptor binding assays, to determine a selected biological activity, one can readily identify operable peptide and protein analogs for use within the methods and compositions of the invention. Operable peptide and protein analogs are typically specifically immunoreactive with antibodies raised to the corresponding native peptide or protein.

An approach for stabilizing solid protein formulations of the invention is to increase the physical stability of purified, e.g., lyophilized, protein. This will inhibit aggregation via hydrophobic interactions as well as via covalent pathways that may increase as proteins unfold. Stabilizing formulations in this context often include polymer-based formulations, for example a biodegradable hydrogel formulation/delivery system. As noted above, the critical role of water in protein structure, function, and stability is well known. Typically, proteins are relatively stable in the solid state with bulk water removed. However, solid therapeutic protein formulations may become hydrated upon storage at elevated humidities or during delivery from a sustained release composition or device. The stability of proteins generally drops with increasing hydration. Water can also play a significant role in solid protein aggregation, for example, by increasing protein flexibility resulting in enhanced accessibility of reactive groups, by providing a mobile phase for reactants. A variety of additives, diluents, bases and delivery vehicles are provided within the invention that enhance protein stability. These reagents are effective as anti-aggregation agents and can serve as a reactant in several deleterious processes such as beta-elimination and hydrolysis.

Protein preparations containing between about 6% to 28% water are the most unstable. Below this level, the mobility of bound water and protein internal motions are low. Above this level, water mobility and protein motions approach those of full hydration. Up to a point, increased susceptibility toward solid-phase aggregation with increasing hydration has been observed in several systems. However, at higher water content, less aggregation is observed because of the dilution effect.

In accordance with these principles, an effective method for stabilizing peptides and proteins against solid-state aggregation for mucosal delivery is to control the water content in a solid formulation and maintain the water activity in the formulation at optimal levels. This level depends on the nature of the protein, but in general, proteins maintained below their “monolayer” water coverage will exhibit superior solid-state stability.

A variety of additives, diluents, bases and delivery vehicles are provided within the invention that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the activity or physical stability of proteins can also be enhanced by various additives to aqueous solutions of the peptide or protein drugs. For example, additives, such as polyols (including sugars), amino acids, proteins such as collagen and gelatin, and various salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as may occur in certain sustained-release formulations of the invention. Proteins such as gelatin and collagen also serve as stabilizing or bulking agents to reduce denaturation and aggregation of unstable proteins in this context. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations for mucosal delivery of aggregation-prone peptides and proteins, wherein the peptide or protein is stabilized in a substantially pure, unaggregated form using a solubilization agent. A range of components and additives are contemplated for use within these methods and formulations. Exemplary of these solubilization agents are cyclodextrins (CDs), which selectively bind hydrophobic side chains of polypeptides. These CDs have been found to bind to hydrophobic patches of proteins in a manner that significantly inhibits aggregation. This inhibition is selective with respect to both the CD and the protein involved. Such selective inhibition of protein aggregation provides additional advantages within the intranasal delivery methods and compositions of the invention. Additional agents for use in this context include CD dimers, trimers and tetramers with varying geometries controlled by the linkers that specifically block aggregation of peptides and protein. Yet solubilization agents and methods for incorporation within the invention involve the use of peptides and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures of the invention.

Effective delivery of biotherapeutic agents via intranasal administration must take into account the decreased drug transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The coordinate administration methods of the instant invention optionally incorporate effective mucolytic or mucus-clearing agents, which serve to degrade, thin, or clear mucus from intranasal mucosal surfaces to facilitate absorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the invention, or as an additive within a combinatorial formulation of the invention, to provide an improved formulation that enhances intranasal delivery of biotherapeutic compounds by reducing the barrier effects of intranasal mucus.

A variety of mucolytic or mucus-clearing agents are available for incorporation within the methods and compositions of the invention. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order: deoxycholate>taurocholate>glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein. α-amylase and β-amylase are representative of this class of enzymes, although their mucolytic effect may be limited. In contrast, bacterial glycosidases which allow these microorganisms to permeate mucus layers of their hosts may have a stronger effect.

For combinatorial use with most biologically active agents within the invention, including peptide and protein therapeutics, non-ionogenic detergents are generally also useful as mucolytic or mucus-clearing agents. These agents typically will not modify or substantially impair the activity of therapeutic polypeptides.

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

Ciliostatic agents, within the methods and compositions of the invention, increase the residence time of mucosally (e.g., intranasally) administered PTH peptide, analogs and mimetics, and other biologically active agents disclosed herein. In particular, within the methods and compositions of the invention, delivery is significantly enhanced in certain aspects by the coordinate administration or combinatorial formulation of one or more ciliostatic agents that function to reversibly inhibit ciliary activity of mucosal cells, to provide for a temporary, reversible increase in the residence time of the mucosally administered active agent(s). For use within these aspects of the invention, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of PTH peptide, analogs and mimetics, and other biologically active agents disclosed herein, without unacceptable adverse side effects.

Certain surface-active agents are readily incorporated within the mucosal delivery formulations and methods of the invention as mucosal absorption enhancing agents. These agents, which may be coordinately administered or combinatorially formulated with PTH peptide proteins, analogs and mimetics, and other biologically active agents disclosed herein, may be selected from a broad assemblage of known surfactants. The mechanisms of action of these various classes of surface-active agents typically include solubilization of the biologically active agent. For proteins and peptides which often form aggregates, the surface active properties of these absorption promoters can allow interactions with proteins such that smaller units such as surfactant coated monomers may be more readily maintained in solution. Examples of other surface-active agents are L-α-phosphatidylcholine didecanoyl (DDPC), polysorbate 80 and polysorbate 20. These monomers are presumably more transportable units than aggregates. Another potential mechanism is the protection of the peptide or protein from proteolytic degradation by proteases in the mucosal environment. Both bile salts and some fusidic acid derivatives reportedly inhibit proteolytic degradation of proteins by nasal homogenates at concentrations less than or equivalent to those required to enhance protein absorption. This protease inhibition may be especially important for peptides with short biological half-lives.

The present invention provides pharmaceutical composition that contains one or more PTH peptides, analogs or mimetics, and/or other biologically active agents in combination with mucosal delivery enhancing agents disclosed herein formulated in a pharmaceutical preparation for mucosal delivery.

In certain aspects of the invention, the combinatorial formulations and/or coordinate administration methods herein incorporate an effective amount of peptides and proteins which may adhere to charged glass thereby reducing the effective concentration in the container. Silanized containers, for example, silanized glass containers, are used to store the finished product to reduce adsorption of the polypeptide or protein to a glass container.

In yet additional aspects of the invention, a kit for treatment of a mammalian subject comprises a stable pharmaceutical composition of one or more PTH peptide compound(s) formulated for mucosal delivery to the mammalian subject wherein the composition is effective for treating or preventing osteoporosis or osteopenia. The kit further comprises a pharmaceutical reagent bottle to contain the one or more PTH peptide compounds. The pharmaceutical reagent bottle is composed of pharmaceutical grade polymer, glass or other suitable material. The pharmaceutical reagent bottle is, for example, a silanized glass bottle. The kit further comprises an aperture for delivery of the composition to a nasal mucosal surface of the subject. The delivery aperture is composed of a pharmaceutical grade polymer, glass or other suitable material. The delivery aperture is, for example, a silanized glass.

A silanization technique combines a special cleaning technique for the surfaces to be silanized with a silanization process at low pressure. The silane is in the gas phase and at an enhanced temperature of the surfaces to be silanized. The method provides reproducible surfaces with stable, homogeneous and functional silane layers having characteristics of a monolayer. The silanized surfaces prevent binding to the glass of polypeptides or mucosal delivery enhancing agents of the present invention.

The procedure is useful to prepare silanized pharmaceutical reagent bottles to hold PTH peptide compositions of the present invention. Glass trays are cleaned by rinsing with double distilled water (ddH₂O) before using. The silane tray is then be rinsed with 95% EtOH, and the acetone tray is rinsed with acetone. Pharmaceutical reagent bottles are sonicated in acetone for 10 minutes. After the acetone sonication, reagent bottles are washed in ddH₂O tray at least twice. Reagent bottles are sonicated in 0.1 M NaOH for 10 minutes. While the reagent bottles are sonicating in NaOH, the silane solution is made under a hood. (Silane solution: 800 mL of 95% ethanol; 96 L of glacial acetic acid; 25 mL of glycidoxypropyltrimethoxy silane). After the NaOH sonication, reagent bottles are washed in ddH₂O tray at least twice. The reagent bottles are sonicated in silane solution for 3 to 5 minutes. The reagent bottles are washed in 100% EtOH tray. The reagent bottles are dried with prepurified N₂ gas and stored in a 100° C. oven for at least 2 hours before using.

Within the compositions and methods of the invention, the PTH peptides, analogs and mimetics, and other biologically active agents disclosed herein may be administered to subjects by a variety of mucosal administration modes, including by oral, rectal, vaginal, intranasal, intrapulmonary, or transdermal delivery, or by topical delivery to the eyes, ears, skin or other mucosal surfaces.

Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069, hereby incorporated by reference. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface-active agent, such as a nonionic surfactant (e.g., polysorbate-80), and water. Another embodiment of the present inviention comprises the drug or drug to be delivered, optionally formulated with methyl-β-cyclodextrin, EDTA, didecanoylphosphatidyl choline, and water. In some embodiments of the present invention, the nasal spray solution further comprises a propellant. The pH of the nasal spray solution is optionally between about pH 3.0 and 6.0, preferably 4.0±0.3. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

To formulate compositions for mucosal delivery within the present invention, the biologically active agent can be combined with various pharmaceutically acceptable additives, as well as a base or carrier for dispersion of the active agent(s). In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage is induced in the nasal mucosa at the site of administration. Generally, the tonicity of the solution is adjusted to a value of about ⅓ to 3, more typically ½ to 2, and most often ¾ to 1.7.

To further enhance mucosal delivery of pharmaceutical agents within the invention, formulations comprising the active agent may also contain a hydrophilic low molecular weight compound as a base or excipient. Such, hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compound include polyol compounds, such as oligo-, di- and monosaccarides such as sucrose, mannitol, sorbitol, lactose, L-arabinose, D-erythrose, D-ribose, D-xylose, D-mannose, trehalose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.) These hydrophilic low molecular weight compounds can be used alone or in combination with one another or with other active or inactive components of the intranasal formulation.

The compositions of the invention may alternatively contain as pharmaceutically acceptable carriers substances as required to approximate physiological conditions, such as tonicity adjusting agents, wetting agents and the like, for example, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

Therapeutic compositions for administering the biologically active agent can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it is desirable to include isotonic agents, for example, sugars, polyalcohols such as mannitol, sorbitol, or sodium chloride in the composition. Prolonged absorption of the biologically active agent can be brought about by including in the composition an agent which delays absorption, for example, monostearate salts and gelatin.

Mucosal administration according to the invention allows effective self-administration of treatment by patients, provided that sufficient safeguards are in place to control and monitor dosing and side effects. Mucosal-administration also overcomes certain drawbacks of other administration forms, such as injections, that are painful and expose the patient to possible infections and may present drug bioavailability problems. For nasal and pulmonary delivery, systems for controlled aerosol dispensing of therapeutic liquids as a spray are well known. In one embodiment, metered doses of active agent are delivered by means of a specially constructed mechanical pump valve, U.S. Pat. No. 4,511,069.

For prophylactic and treatment purposes, the biologically active agent(s) disclosed herein may be administered to the subject intranasally once daily. In this context, a therapeutically effective dosage of the PTH peptide may include repeated doses within a prolonged prophylaxis or treatment regimen that will yield clinically significant results to alleviate or prevent osteoporosis or osteopenia. Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

The actual dosage of biologically active agents will of course vary according to factors such as the disease indication and particular status of the subject (e.g., the subject's age, size, fitness, extent of symptoms, susceptibility factors, etc), time and route of administration, other drugs or treatments being administered concurrently, as well as the specific pharmacology of the biologically active agent(s) for eliciting the desired activity or biological response in the subject. Dosage regimens may be adjusted to provide an optimum prophylactic or therapeutic response. A therapeutically effective amount is also one in which any toxic or detrimental side effects of the biologically active agent are outweighed in clinical terms by therapeutically beneficial effects. A non-limiting range for a therapeutically effective amount of a PTH peptide within the methods and formulations of the invention is 0.7 μg/kg to about 25 μg/kg. To treat osteoporosis or osteopenia, an intranasal dose of PTH peptide is administered at dose high enough to promote the increase in bone mass but low enough so as not to induce any unwanted side-effects such as nausea. A preferred intranasal dose of PTH (1 -34) is about 1 to about 10 μg/kg weight of the patient, most preferably from about 1.5 to about 3 μg/kg weight of the patient. In a standard dose a patient will receive about 50 to about 1600 μg, more preferably about between 75 to 800 μg, most preferably 100 μg, 150 μg, or 200 μg to about 400 μg. Alternatively, a non-limiting range for a therapeutically effective amount of a biologically active agent within the methods and formulations of the invention is between about 0.001 pmol to about 100 pmol per kg body weight, between about 0.01 pmol to about 10 pmol per kg body weight, between about 0.1 pmol to about 5 pmol per kg body weight, or between about 0.5 pmol to about 1.0 pmol per kg body weight. Per administration, it is desirable to administer at least one microgram of the biologically active agent (e.g., one or more PTH peptide proteins, analogs and mimetics, and other biologically active agents), more typically between about 10 μg and 5.0 mg, and in certain embodiments between about 100 μg and 1.0 or 2.0 mg to an average human subject. For certain oral applications, doses as high as 0.5 mg per kg body weight may be necessary to achieve adequate plasma levels. It is to be further noted that for each particular subject, specific dosage regimens should be evaluated and adjusted over time according to the individual need and professional judgment of the person administering or supervising the administration of the permeabilizing peptide(s) and other biologically active agent(s). An intranasal dose of a parathyroid hormone will range from 50 μg to 1600 μg of parathyroid hormone, preferably 75 μg to 800 μg, more preferably 100 μg to 400 μg with a most preferred dose being between 100 μg to 200μg with 150 μg being a dose that is considered to be highly effective. Repeated intranasal dosing with the formulations of the invention, on a schedule ranging from about 0.1 to 24 hours between doses, preferably between 0.5 and 24.0 hours between doses, will maintain normalized, sustained therapeutic levels of PTH peptide to maximize clinical benefits while minimizing the risks of excessive exposure and side effects. The goal is to mucosally deliver an amount of the PTH peptide sufficient to raise the concentration of the PTH peptide in the plasma of an individual to promote increase in bone mass.

Dosage of PTH agonists such as parathyroid hormone may be varied by the attending clinician or patient, if self administering an over the counter dosage form, to maintain a desired concentration at the target site.

In an alternative embodiment, the invention provides compositions and methods for intranasal delivery of PTH peptide, wherein the PTH peptide compound(s) is/are repeatedly administered through an intranasal effective dosage regimen that involves multiple administrations of the PTH peptide to the subject during a daily or weekly schedule to maintain a therapeutically effective elevated and lowered pulsatile level of PTH peptide during an extended dosing period. The compositions and method provide PTH peptide compound(s) that are self-administered by the subject in a nasal formulation between one and six times daily to maintain a therapeutically effective elevated and lowered pulsatile level of PTH peptide during an 8 hour to 24 hour extended dosing period.

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more PTH peptide proteins, analogs or mimetics, and/or other biologically active agents in combination with mucosal delivery enhancing agents disclosed herein formulated in a pharmaceutical preparation for mucosal delivery.

The intranasal formulations of the present invention can be administered using any spray bottle (i.e., a bottle with an actuator, spray pump). An example of a nasal spray bottle is the, “Nasal Spray Pump w/ Safety Clip”, which delivers a dose of 0.1 mL per squirt and has a diptube length of 36.05 mm (Pfeiffer of America, Princeton, N.J.). Intranasal doses of a PTH peptide such as parathyroid hormone can range from 0.1 μg/kg to about 1500 μg/kg. When administered in an intranasal spray, it is preferable that the particle size of the spray is between 10-100 μm (microns) in size, preferably 20-100 μm in size.

We have discovered that the parathyroid hormone peptides can be administered intranasally using a nasal spray or aerosol. This is surprising because many proteins and peptides have been shown to be sheared or denatured due to the mechanical forces generated by the actuator in producing the spray or aerosol. In this area the following definitions are useful.

1. Aerosol—A product that is packaged under pressure and contains therapeutically active ingredients that are released upon activation of an appropriate valve system.

2. Metered aerosol—A pressurized dosage form comprised of metered dose valves, which allow for the delivery of a uniform quantity of spray upon each activation.

3. Powder aerosol—A product that is packaged under pressure and contains therapeutically active ingredients in the form of a powder, which are released upon activation of an appropriate valve system.

4. Spray aerosol—An aerosol product that utilizes a compressed gas as the propellant to provide the force necessary to expet the product as a wet spray; it generally applicable to solutions of medicinal agents in aqueous solvents.

5. Spray—A liquid minutely divided as by a jet of air or steam. Nasal spray drug products contain therapeutically active ingredients dissolved or suspended in solutions or mixtures of excipients in nonpressurized dispensers.

6. Metered spray—A non-pressurized dosage form consisting of valves that allow the dispensing of a specified quantity of spray upon each activation.

7. Suspension spray—A liquid preparation containing solid particles dispersed in a liquid vehicle and in the form of course droplets or as finely divided solids.

The fluid dynamic characterization of the aerosol spray emitted by metered nasal spray pumps as a drug delivery device (“DDD”). Spray characterization is an integral part of the regulatory submissions necessary for Food and Drug Administration (“FDA”) approval of research and development, quality assurance and stability testing procedures for new and existing nasal spray pumps.

Thorough characterization of the spray's geometry has been found to be the best indicator of the overall performance of nasal spray pumps. In particular, measurements of the spray's divergence angle (plume geometry) as it exits the device; the spray's cross-sectional ellipticity, uniformity and particle/droplet distribution (spray pattern); and the time evolution of the developing spray have been found to be the most representative performance quantities in the characterization of a nasal spray pump. During quality assurance and stability testing, plume geometry and spray pattern measurements are key identifiers for verifying consistency and conformity with the approved data criteria for the nasal spray pumps.

Definitions

Plume Height—the measurement from the actuator tip to the point at which the plume angle becomes non-linear because of the breakdown of linear flow. Based on a visual examination of digital images, and to establish a measurement point for width that is consistent with the farthest measurement point of spray pattern, a height of 30 mm is defined for this study

Major Axis—the largest chord that can be drawn within the fitted spray pattern that crosses the COMw in base units (mm)

Minor Axis—the smallest chord that can be drawn within the fitted spray pattern that crosses the COMw in base units (mm)

Ellipticity Ratio—the ratio of the major axis to the minor axis

D₁₀—the diameter of droplet for which 10% of the total liquid volume of sample consists of droplets of a smaller diameter (μm)

D₅₀—the diameter of droplet for which 50% of the total liquid volume of sample consists of droplets of a smaller diameter (μm), also known as the mass median diameter

D₉₀—the diameter of droplet for which 90% of the total liquid volume of sample consists of droplets of a smaller diameter (μm)

Span—measurement of the width of the distribution, the smaller the value, the narrower the distribution. Span is calculated as (D₉₀−D₁₀)/D₅₀.

% RSD—percent relative standard deviation, the standard deviation divided by the mean of the series and multiplied by 100, also known as % CV.

A nasal spray device can be selected according to what is customary in the industry or acceptable by the regulatory health authorities. One example of a suitable device is described in described in U.S. application Ser. No. 10/869,649 (S. Quay and G. Brandt: Compositions and methods for enhanced mucosal delivery of Y2 receptor-binding peptides and methods for treating and preventing obesity, filed Jun. 16, 2004).

To treat osteoporosis or osteopenia, an intranasal dose of a PTH peptide parathyroid hormone is administered at dose high enough to promote an increase in bone mass but low enough so as not to induce any unwanted side-effects such as nausea. A preferred intranasal dose of a PTH peptide such as parathyroid hormone (1-34) is about 3 μg-10 μg/kg weight of the patient, most preferably about 6 μg/kg weight of the patient. In a standard dose a patient will receive 50 μg to 800 μg, more preferably about between 100 μg to 400 μg, most preferably 150 μg to about 200 μg. A PTH peptide such as parathyroid hormone (1-34) is preferably administered once a day.

The following examples are provided by way of illustration, not limitation.

### EXAMPLES

The exemplary discosure of copending U.S. Application Ser. Nos. 11/246,406 and 11/246,450 filed Oct. 6, 2005 and U.S. Application Ser. No. 11/126,996 filed May 10, 2005 are hereby incorporated by reference in their entirety.

### Example 1

### Stability

A PTH formulation will be supplied as a liquid in a bottle for intranasal administration via an actuator. Formulations containing 1-10 mg/mL PTH at pH 4.0-4.5 were tested for “as-sold” stability. “As-sold” stability studies are defined as those studies involving formulation stored within a closed (i.e., capped) bottle, placed at specific storage or accelerated temperature conditions for specified amounts of time. Formulation excipients were selected from the group consisting of PTH; methyl-β-cyclodextrin (M-β-CD); ethylenediaminetetraacetic acid (EDTA); didecanoylphosphatidyl choline (DDPC); chlorobutanol (CB); sodium benzoate (NaBZ), polysorbate 80 (Tween 80), and sorbitol. The initial pH of the formulations was adjusted to pH 4.0 or 4.5 with sodium hydroxide or hydrochloric acid, as necessary. The formulations that were tested are shown in Table 1.

TABLE 1
Composition of various intranasal PTH formulations

| Formulation # | Composition |
|---|---|
| 1 | 1 mg/mL PTH, 5 mg/mL preservative (CB), 45 mg/mL |
| | M-β-CD, 1 mg/mL DDPC, 1 mg/mL EDTA, |
| | 26 mg/mL sorbitol, pH ˜4.0 |
| 2 | 1.5 mg/mL PTH, 5 mg/mL preservative (CB), 45 mg/mL |
| | M-β-CD, 1 mg/mL |
| | DDPC, 1 mg/mL EDTA, 26 mg/mL sorbitol, pH ˜4.0 |
| 3 | 2 mg/mL PTH, 5 mg/mL preservative (CB or NaBz), |
| | 45 mg/mL M-β- |
| | CD, 1 mg/mL DDPC, 1 mg/mL EDTA, 16.7 mg/mL |
| | sorbitol, pH ˜4.0 or 4.5 |
| 4 | 3 mg/mL PTH, 5 mg/mL preservative (CB), 1 mg/mL |
| | polysorbate 80, 31 mg/mL sorbitol, pH ˜4.0 |
| 5 | 4 mg/mL PTH, 5 mg/mL preservative (CB), 1 mg/mL |
| | polysorbate 80, 31 mg/mL sorbitol, pH ˜4.0 |
| 6 | 5 mg/mL PTH, 5 mg/mL preservative (CB or |
| | NaBz), 1 mg/mL polysorbate 80, 27.2 mg/mL |
| | sorbitol, pH ˜4 |
| 7 | 10 mg/mL PTH, 5 mg/mL preservative (CB or |
| | NaBz), 1 mg/mL polysorbate 80, 27.2 mg/mL sorbitol, |
| | pH ˜4 |

The reported storage conditions for injectable Forteo (ingredients: teriparatide, glacial acetic acid, sodium acetate, mannitol, m-cresol, and water) is 2-8° C. for up to 28 days (four weeks). The storage stability of PTH formulations 1, 3, 4, and 7 was monitored at regular intervals by determining the remaining percentage of PTH relative to initial using HPLC. All four formulations used in the stability studies included CB as preservative and were at a pH of 4.0. The results in Tables 2 and 3 show PTH intranasal formulations 1, 3, 4, and 7 may be safely stored at 5° C. and 25° C. for at least four weeks without a significant decrease in stability. Formulations 1, 3, 4, and 7 remained stable for at least 24 weeks when stored at 5° C. Formulation #7 was the most stable of the tested formulations at 5° C. and 25° C. Storage conditions of PTH intranasal formulations at 5° C. for at least 24 weeks is longer than the current recommended storage conditions for injectable Forteo.

TABLE 2
Percent Stability of PTH Formulations at 5° C.

| | Formulation # (5° C.) | | | |
|---|---|---|---|---|
| Time (weeks) | 1 | 3 | 4 | 7 |
| Initial | 100 ± 1.6 | 100 ± 2.3 | 100 ± 0.4 | 100 ± 2.2 |
| 2 | 101.5 ± 1.1 | 99.8 ± 1.9 | 97.5 ± 0.7 | 100.5 ± 1.3 |
| 4 | 98.1 ± 0.9 | 96.5 ± 3.0 | 100 ± 0.6 | 99.3 ± 2.0 |
| 8 | 96.5 ± 3.2 | 98.2 ± 1.7 | 95.7 ± 1.0 | 95.1 ± 6.6 |
| 12 | 97.4 ± 4.1 | 98.8 ± 2.5 | 97.7 ± 1.5 | 103.3 ± 2.3 |
| 24 | 95.2 ± 0.9 | 94.8 ± 1.2 | 97.3 ± 0.5 | 100.6 ± 2.5 |

TABLE 3
Percent Stability of PTH Formulations at 25° C.

| | Formulation # (25° C.) | | | |
|---|---|---|---|---|
| Time (weeks) | 1 | 3 | 4 | 7 |
| Initial | 100 ± 1.6 | 100 ± 2.3 | 100 ± 0.4 | 100 ± 2.2 |
| 2 | 98.3 ± 1.1 | 98.2 ± 2.3 | 97.5 ± 0.2 | 99.7 ± 1.3 |
| 4 | 96.4 ± 1.6 | 93.2 ± 2.2 | 96.2 ± 2.3 | 97.7 ± 1.3 |
| 8 | 91.1 ± 5.2 | 89.6 ± 8.3 | 90.0 ± 0.4 | 92.8 ± 2.8 |
| 12 | 85.4 ± 7.8 | 89.8 ± 4.0 | 94.5 ± 1.0 | 97.1 ± 1.5 |
| 24 | 80.9 ± 1.0 | 81.7 ± 1.2 | 83.9 ± 1.1 | 87.7 ± 1.6 |

Further characterization of the stability of PTH formulations without buffer was conducted at 30° C. (Table 4), 40° C. (Table 5), and 50° C. (Table 6). The percent PTH remaining from initial was determined at 1, 2, 3, and 4 week timepoints. The 30° C. data without buffer is compared to the injectable formulation data containing buffer from U.S. Pat. No. 6,770,623 (the '623 formulation). The '623 formulation contained 0.1 mg/mL rhPTH (1-34), 50 mg/mL mannitol, 2.5 mg/mL m-cresol, 0.52 mg/mL acetic acid and 0.12 mg/mL sodium acetate. Formulations 1 and 4 without a buffer at 30° C. had stability similar to the '623 formulation with buffer at 30° C. At 50° C., Formulations 1, 3, 4 and 7 have a greater stability than the '623 formulation. Formulation 7 was the most stable compared to other formulations tested at 40° C. and 50° C.

TABLE 4
Percent Stability with and without buffer at 30° C.

| | With buffer | | | | Without buffer | |
|---|---|---|---|---|---|---|
| | 20 mM | 10 mM | | | Formu- | |
| Time | acetate | acetate | | | lation | Formulation |
| (weeks) | ('623) | ('623) | '623 1 | '623 2 | # 1 | # 4 |
| Initial | 100 | 100 | 100 | 100 | 100 | 100 |
| 1 | 96 | 94 | 100 | — | 101 ± 4.5 | 114 ± 1.5 |
| 2 | 94 | 92 | 96 | 100 | 73.7 ± 2.0 | 105.5 ± 4.3 |
| 3 | 90 | 93 | 97 | — | 94.7 ± 1.8 | 106.2 ± 1.5 |
| 4 | — | 81 | 96 | 96 | 93.8 | 101.6 |

TABLE 5
Percent Stability of PTH formulations at 40° C.

| | Formulation # (40° C.) | | | |
|---|---|---|---|---|
| Time (weeks) | 1 | 3 | 4 | 7 |
| Initial | 100 ± 1.6 | 100 ± 2.3 | 100 ± 0.4 | 100 ± 2.2 |
| 1 | 90.2 ± 1.3 | 92.9 ± 1.5 | 93.9 ± 0.8 | 96.5 ± 1.6 |
| 2 | 80.7 ± 2.8 | 86.1 ± 1.1 | 83.9 ± 0.8 | 88.0 ± 1.3 |
| 4 | 66.9 ± 1.8 | 70.9 ± 1.6 | 70.3 ± 2.1 | 71.7 ± 2.2 |

TABLE 6
Percent Stability with and without buffer at 50° C.

| | Formulations | | | | | | | |
|---|---|---|---|---|---|---|---|---|
| | With buffer | | Formulation # | | | | | |
| | 20 mM | 10 mM | 0.9% | | | | | |
| Time | acetate | acetate | NaCl | Water | | | | |
| (weeks) | ('623) | ('623) | ('623) | ('623) | 1 | 3 | 4 | 7 |
| Initial | 100 | 100 | 100 | 100 | 100 ± 1.6 | 100 ± 2.3 | 100 ± 0.4 | 100 ± 2.2 |
| 1 | 84 | 80 | 81 | 74 | 88.9 ± 2.4 | 89.6 ± 3.0 | 88.6 ± 0.2 | 91.6 ± 1.6 |
| 2 | 67 | 71 | 58 | 55 | 76.6 ± 1.8 | 75.9 ± 2.2 | 73.5 ± 0.5 | 76.7 ± 2.9 |
| 4 | — | — | — | — | 54.3 ± 1.2 | 54.5 ± 4.4 | 52.0 ± 0.9 | 56.7 ± 0.8 |

PTH formulations 1 and 4 were also tested for in-use and spray stability at both 5° C. and 30° C. storage temperatures over a 29-day period. Results include % Peptide Recover and % Total Peptide Impurity. “In-use” studies are those in which. an actuator is present and the bottles were primed five times initially, and then actuated once daily by hand after subjecting to the storage temperatures. All bottles were returned to the 5° C. and 30° C. stability chamber after 30 minute exposure to room temperature. All bottles were actuated daily, and the actuated samples were collected and stored at −20° C. until scheduled for HPLC measurements. HPLC measurements are scheduled for in-use (i.e., in the bottle with an actuator present) and spray (i.e., measured from the spray produced by the actuator in the bottle) samples at Day 0, Day 8, Day 15, Day22 and Day 29. The HPLC measurements for stability are shown in Table 7 (% Peptide Recovery) and Table 8 (% Impurity).

TABLE 7
In-use and Spray % Peptide Recovery at 5° C. and 30° C.

| Time Point | Formulation | Formulation | Formulation | Formulation |
|---|---|---|---|---|
| (days) | #1 | #4 | #1 | #4 |
| | In-use 5° C. | | Spray 5° C. | |
| 0 | 100.0 | 100.0 | 100.0 | 100.0 |
| 15 | 94.2 | 97.8 | 93.9 | 97.3 |
| 22 | 93.8 | 100.1 | 103.0 | 107.9 |
| 29 | 99.3 | 105.3 | 32.9 | 106.0 |
| | In-use 30° C. | | Spray 30° C. | |
| 0 | 100.0 | 100.0 | 100.0 | 100.0 |
| 8 | 103.3 | 107.0 | 109.7 | 110.6 |
| 15 | 84.7 | 99.3 | 130.8 | 103.8 |
| 22 | 98.8 | 103.0 | 99.6 | 101.9 |
| 29 | 94.3 | 97.8 | 34.7 | 102.3 |

TABLE 8
In-use and Spray Total Peptide Impurity at 5° C. and 30° C.

| Time | Formu- | Formu- | Formu- | Formu- | Formu- | Formu- |
|---|---|---|---|---|---|---|
| Point | lation | lation | lation | lation | lation | lation |
| (days) | #1 | #4 | #1 | #4 | #1 | #4 |
| | As-sold 5° C. | | In-use 5° C. | | Spray 5° C. | |
| 0 | 0.9 | 0.4 | 0.5 | 0.3 | 0.5 | 0.5 |
| 8 | 0.9 | 0.7 | 0.7 | 0.5 | 1.1 | 0.7 |
| 15 | 0.9 | 0.4 | 0.7 | 0.5 | 0.8 | 0.5 |
| 22 | 0.8 | 0.6 | 1.1 | 1.4 | 1.6 | 1.3 |
| 29 | 1.7 | 0.7 | 2.0 | 1.3 | 3.8 | 1.6 |
| | As-sold 30° C. | | In-use 30° C. | | Spray 30° C. | |
| 0 | 0.9 | 0.3 | 0.5 | 0.3 | 0.5 | 0.5 |
| 8 | 1.7 | 1.5 | 2.0 | 1.5 | 3.0 | 1.5 |
| 15 | 1.8 | 1.5 | 1.8 | 1.5 | 3.5 | 2.0 |
| 22 | 4.6 | 3.2 | 4.5 | 3.2 | 5.0 | 3.7 |
| 29 | 6.2 | 5.0 | 6.5 | 5.0 | 15.4 | 5.1 |

As-sold, in-use and spray stability studies showed that Formulation #4 (containing polysorbate 80) was more stable than Formulation #1 (containing EDTA). Further studies confirmed that EDTA alone or in combination with polysorbate 80 was inferior to PTH formulations without EDTA. Formulations with EDTA alone caused precipitation and gelling. When EDTA was added in combination with other excipients an increased instability was observed. Stability studies showed that polysorbate 80 alone and in combination with other excipients enhanced stability. Addition of ethanol to the PTH formulations did not enhance stability. NaBz contributed to turbidity of the PTH formulations while results showed that CB was the preferred preservative for a stable PTH formulation.

### Example 2

### pH Stability

The following formulations were tested for pH stability.

TABLE 9

| | Conc. (mg/ml) | | | | | | | | | | |
|---|---|---|---|---|---|---|---|---|---|---|---|
| | | | | Polysorbate | | | Glacial | Sodium | | | |
| Diluent | M-β-CD | DDPC | EDTA | 80 | CB | Sorbitol | acetic acid | acetate | Mannitol | m-Cresol | pH |
| Forteo | 0 | 0 | 0 | 0 | 0 | 0 | 0.41 | 0.1 | 45.4 | 3 | 4.0 |
| MBCD | 45 | 1 | 1 | 0 | 2.5 | 29 | 0 | 0 | 0 | 0 | 4.0 |
| Tween | 0 | 0 | 0 | 1 | 2.5 | 36 | 0 | 0 | 0 | 0 | 4.0 |

Solutions without PTH were first tested by pH titration. All three diluents had a pH value of 4.0 before the pH titration. The pH shifts resulting from the addition of base to the Forteo, MBCD and Tween formulations containing 1-4 mg/mL PTH and stored without buffer maintain a pH of 4.0 to 4.2 after at least 8 weeks of storage at 5° C. and 25° C. (Table 10). These data show that the PTH formulation composition stably maintains pH without a buffer.

TABLE 10
pH stability for MBCD and Tween Formulations at 5° C. and 25° C.

| | pH | | | | | | |
|---|---|---|---|---|---|---|---|
| | 5° C. | | | 25° C. | | | |
| | In- | | | In- | | | |
| | i- | 4 | 8 | i- | | | |
| Formulations | tial | weeks | weeks | tial | 2 weeks | 4 weeks | 8 weeks |
| 1 mg/mL PTH | 4.0 | 4.1 | 4.0 | 4.0 | 4.0 | 4.1 | 4.1 |
| MBCD* | | | | | | | |
| 2 mg/mL PTH | 4.0 | 4.0 | 4.0 | 4.0 | 4.0 | 4.1 | 4.0 |
| MBCD* | | | | | | | |
| 2 mg/mL PTH | 4.0 | 4.2 | 4.1 | 4.0 | 4.1 | 4.1 | 4.1 |
| Tween* | | | | | | | |
| 4 mg/mL PTH | 4.0 | 4.1 | 4.1 | 4.0 | 4.1 | 4.1 | 4.1 |
| Tween* | | | | | | | |

*CB at 2.5 mg/mL

### Example 3

### Pharmacokinetics (PK) in Human Subjects

The absorption and safety of the PTH nasal spray formulations of the invention were evaluated at two dose levels. The bioavailability of FORSTEO (Eli Lilly UK) given subcutaneously was compared with that of two PTH nasal spray formulations of the invention at two dose levels. PTH Nasal Spray will be supplied to the clinic as a liquid in a bottle for intranasal administration via an actuator. Details for formulation compositions between 1.0 and 4.0 mg/mL PTH strengths are shown in Table 1. For the PK studies, Formulations 3, 6, and 7 included NaBz as the preservative. Formulation 3 had a pH of 4.5, while all other formulations were at pH 4.0.

The PTH solution is provided in a multi-unit dose container to deliver a metered dose of 0.1 mL of drug product per actuation. Hydrochloric acid is added for pH adjustment to meet target pH of 4.0±0.2 or 4.5±0.2, as appropriate. The stability of the formulations was monitored at regular intervals.

This study was a single-site, open-label, active controlled, 5 period crossover, dose ranging study involving 6 healthy male and 6 healthy female volunteers. The commercially available formulation of teriparatide, FORSTEO was the active control. The five study periods were as follows:

Period 1: All subjects received FORSTEO (injection) 20 μg subcutaneously.

Period 2: All subjects received 500 μg intranasal dose of teriparatide, 100 microliter spray of intranasal formulation as described in Example 1, Formulation #6, Table 1.

Period 3: All subjects received 200 μg intranasal dose of teriparatide, 100 microliter spray of intranasal formulation as described in Example 1, Formulation #3 Table 1.

Period 4: All subjects received a 1000 μg intranasal dose of teriparatide, 100 microliter spray of intranasal formulation as described in Example 1, Formulation #6 Table 1.

Period 5: All subjects received a 400 μg intranasal dose of teriparatide, 2×100 microliter spray of intranasal formulation as described in Example 1, Formulation #3 Table 1.

Blood samples for PK were collected at 0 (i.e., pre-dose), 5, 10, 15, 30, 45, 60, 90 minutes and 2, 3, and 4 hours post-dose and analyzed using a validated method. Because the bioassay is fully cross reactive with endogenous PTH(1-84), all data was corrected for pre-dose values. When this correction resulted in a negative post-dose value, all such negative values were set to ‘missing’. Values reported as <LLOQ were set to half LLOQ in order to evaluate PK and change from baseline. Standard pharmacokinetic parameters, including AUCₗₐₛₜ, AUC_{inf}, Cₘₐₓ, t_{1/2}, tₘₐₓ, and Kₑ were calculated using WinNonlin. Intra-subject variability of the pharmacokinetic profiles was evaluated for the test versus the reference using analysis of variance methods. An analysis of variance (ANOVA) was performed based on a 2-period design and incorporating a main effect term for each of the two products under consideration (Snedecor GW and Cochran WG, One-Way Classifications—Analysis of Variance. In: Statistical Methods, 6^{th} ed.: Iowa State University Press, Ames, Iowa, (1967) pp. 258-98). (Subject (Sequence) was a random effect in the model with all others fixed.) A separate model was created for each dose of teriparatide nasal spray versus the reference. The 90% confidence intervals were generated for the ratio of test dose/reference with respect to Cₘₐₓ, AUCₗₐₛₜ, and AUC_{inf}. These values were natural log (ln)-transformed prior to analysis. The corresponding 90% confidence intervals for the geometric mean ratio were obtained by taking the antilog of the 90% confidence intervals for the difference between the means on the log scale. These analyses were not performed to demonstrate bioequivalence but were for informational purposes only. As a result, no adjustment to the confidence level for each of the paired comparisons was made to account for multiplicity of analysis. This study is hypothesis-generating only. For tₘₐₓ, the analyses were run using Wilcoxon's signed-rank test (Steinijans VW and Diletti E (1983) Eur J Clin Pharmacol. 24:127-36) to determine if differences existed between a given test group and the reference group.

For each subject, the following PK parameters were calculated, whenever possible, based on the plasma concentrations of teriparatide for each test article, according to the model independent approach:

Cₘₐₓ Maximum observed concentration

tₘₐₓ Time to maximum concentration

AUCₗₐₛₜ Area under the concentration-time curve from time 0 to the time of last measurable concentration, calculated by the linear trapezoidal rule.

The following parameters were calculated when the data permited accurate estimation of these parameters:

AUC_{inf} Area under the concentration-time curve extrapolated to infinity, calculated using the formula:

AUC_{inf}=AUCₗₐₛₜ+Cₜ/Kₑ where Cₜ is the last measurable concentration and Kₑ is the apparent terminal phase rate constant.

Kₑ Apparent terminal phase rate constant, where Kₑ is the magnitude of the slope of the linear regression of the log concentration versus time profile during the terminal phase.

t_{1/2} Apparent terminal phase half-life (whenever possible), where t_{1/2}=(ln2)/Kₑ. All data was corrected for pre-dose values. When this correction resulted in a negative post-dose value, all such negative values were set to ‘missing’. Values reported as <LLOQ were set to half LLOQ in order to evaluate pK and change from baseline. Actual (not nominal) sampling times were used in the calculation of all PK parameters.

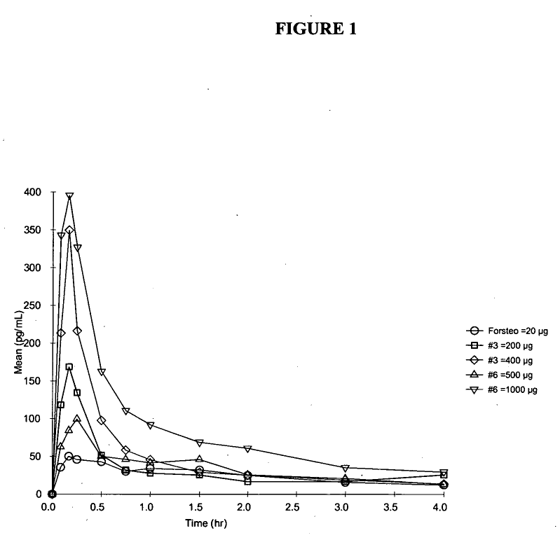

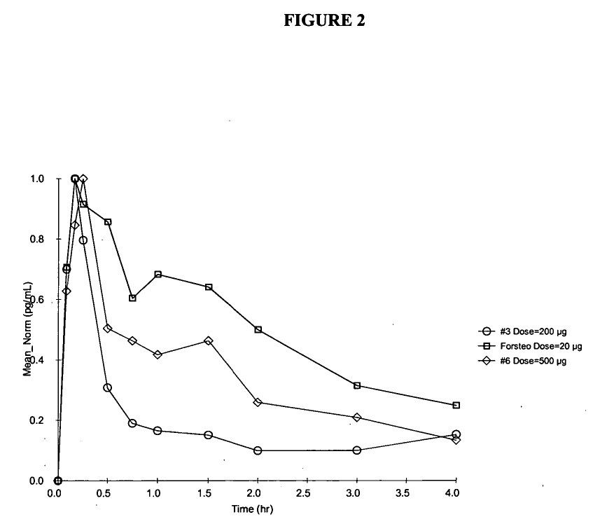

FIGS. 1 and 2 show the mean plasma concentration versus time for periods 1-5, and the ratio of Cₘₐₓ to mean, low dose formulations versus Forsteo, respectively.

A summary of arithmetic mean pharmacokinetic parameters for each formulation and dose of teriparatide are presented in Table 11. The mean tₘₐₓ was 0.68 versus 0.57 and 0.17 hours for the FORSTEO and low dose nasal formulations of Formulation #3 and #6. The Cₘₐₓ was 1.6 and 2.4 fold higher than FORSTEO for each low dose formulation. The AUCₗₐₛₜ was 1.23 and 1.45 fold higher than FORSTEO for each low dose formulation.

TABLE 11
Arithmetic Mean Pharmacokinetic Parameters by Formulation and Dose

| | | | | AUClast | AUCinf | | |
|---|---|---|---|---|---|---|---|
| | Dose | Tmax | Cmax | (hr * | (hr * | t½ | Ke |
| Formulation | (μg) | (hr) | (pg/mL) | pg/mL) | pg/mL) | (hr) | (1/hr) |
| FORSTEO | 20 | 0.68 | 70.80 | 85.92 | 132.12 | 1.57 | 0.638 |
| (injection) | | | | | | | |
| Formulation | 500 | 0.57 | 112.72 | 106.08 | 195.69 | 1.38 | 0.610 |
| #6 | | | | | | | |
| Formulation | 1000 | 0.46 | 405.57 | 335.20 | 412.47 | 1.03 | 0.782 |
| #6 | | | | | | | |
| Formulation | 200 | 0.17 | 172.72 | 125.07 | 269.60 | 3.10 | 0.720 |
| #3 | | | | | | | |
| Formulation | 400 | 0.18 | 349.62 | 206.02 | 238.26 | 1.12 | 1.097 |
| #3 | | | | | | | |

In addition, the tₘₐₓ results for each formulation were compared to the FORSTEO control using a simple Wilcoxon signed-rank test. The results (as p-values) are given in Table 12.

TABLE 12
Comparison of Tₘₐₓ - FORSTEO and Nasal Formulations

| | p-value from Wilcoxon |
|---|---|
| | Signed-Rank |
| Comparison of Tₘₐₓ | Test |
| FORSTEO vs. Formulation #6, 500 μg | 0.75 |
| FORSTEO vs. Formulation #6, 1000 μg | 0.53 |
| FORSTEO vs. Formulation #3, 200 μg | 0.10 |
| FORSTEO vs. Formulation #3, 400 μg | 0.24 |

Thus, there does not appear to be differences in the tₘₐₓ values among the formulations with respect to FORSTEO.

The 90% confidence intervals for the comparison of the given formulation and the FORSTEO control for the ratios of Cₘₐₓ, AUCₗₐₛₜ and AUC_{inf} was calculated. The comparisons of each product with FORSTEO were done on a pairwise basis, but no adjustment for multiple testing was included because of the nature of this study.

A summary of clearance rates using the non-compartmental model are presented in Table 13:

TABLE 13
Summary of Clearance Rates

| Formulation | Dose (μg) | Mean (mL/hr) | SD |
|---|---|---|---|
| Formulation #3 | 200 | 1366234.334 | 988398.4 |
| Formulation #3, | 400 | 2527292.583 | 1701658 |
| FORSTEO | 20 | 267446.6298 | 263855.3 |
| Formulation #6 | 500 | 4793716.136 | 4380229 |
| Formulation #6 | 1000 | 3359436.634 | 1665618 |

A summary of percent coefficient of variation for each formulation and dose of teriparatide are presented in Table 14. Based on Cₘₐₓ and AUCₗₐₛₜ, the % CV is lower for Formulation #3 than Formulation #6 or FORSTEO.

TABLE 14
Percent Coefficient of Variation by Formulation and Dose

| | Dose | Tmax | Cmax | AUClast | AUCinf |
|---|---|---|---|---|---|
| Formulation | (ug) | (hr) | (pg/mL) | (hr * pg/mL) | (hr * pg/mL) |
| FORSTEO | 20 | 165.29 | 51.76 | 66.46 | 62.30 |
| Formulation #6 | 500 | 142.48 | 78.71 | 92.76 | 83.41 |
| Formulation #6 | 1000 | 176.56 | 67.06 | 75.55 | 71.56 |
| Formulation #3 | 200 | 24.72 | 38.78 | 61.55 | 82.28 |
| Formulation #3 | 400 | 21.20 | 48.78 | 55.98 | 68.04 |

A summary of percent relative-bioavailability comparing each formulation to the FORSTEO product based on AUCₗₐₛₜ are presented in Table 15. The bioavailability of the Formulation #3 is 12-15%, whereas Formulation #3 is approximately 5-8%.

TABLE 15
Relative Bioavailability Compared with FORSTEO by Formulation
and Dose

| | Dose | |
|---|---|---|
| Formulation | (ug) | % Bioavailability |
| Formulation #6 | 500 | 4.9 |
| Formulation #6 | 1000 | 7.8 |
| Formulation #3 | 200 | 14.6 |
| Formulation #3 | 400 | 12.0 |

An exploratory compartmental analysis using WinNonLin 5.0 was conducted to compare the absorption coefficient and elimination coefficient for each formulation. A mixed model analysis of variance on both the Ka and the Ke data, where the subject was included as the random variable was performed, and these results were subanalyzed using the Tukey-Kramer multiple comparison procedure. The individual Ka and Ke data are presented in Table 16. The nasal absorption rates were not significantly different compared to FORSTEO (p=0.50), however the elimination rate for high dose nasal Formulation #3 was significantly faster (p=0.02) than FORSTEO. This is also observed when looking at the ratio of mean Cₘₐₓ to each individual time point per low dose formulation.

TABLE 16
Absorption Coefficient and Elimination Coefficient for Each Formulation

| | | Dose | | Mean | | |
|---|---|---|---|---|---|---|
| Coefficient | Formulation | (μg) | N | (1/hr) | SD | CV % |
| Ka | FORSTEO | 20 | 11 | 11.99 | 7.00 | 58.34 |
| Ka | Formulation #6 | 500 | 8 | 6.95 | 4.83 | 69.46 |
| Ka | Formulation #6 | 1000 | 7 | 10.43 | 7.49 | 71.81 |
| Ka | Formulation #3 | 200 | 6 | 11.02 | 5.29 | 48.05 |
| Ka | Formulation #3 | 400 | 7 | 8.81 | 3.19 | 36.27 |
| Ke | FORSTEO | 20 | 11 | 1.04 | 0.86 | 83.50 |
| Ke | Formulation #6 | 500 | 8 | 1.40 | 1.70 | 121.57 |
| Ke | Formulation #6 | 1000 | 7 | 1.83 | 2.50 | 136.49 |
| Ke | Formulation #3 | 200 | 6 | 2.74 | 2.24 | 81.85 |
| Ke | Formulation #3 | 400 | 7 | 4.08 | 2.35 | 57.69 |

Based on the pharmacokinetic parameters, both nasal formulations had a greater Cₘₐₓ and AUC as compared to FORSTEO. The tₘₐₓ occurred sooner after dosing for the nasal formulations, particularly for Formulation #3. The absorption rates were not significantly different among the nasal and subcutaneous formulations (p=0.5), but elimination rates were faster particularly for the low dose Formulation #3 (p=0.02). However, a t_{1/2} of approximately 1 hour was very similar for the nasal formulations compared to FORSTEO, except for the low dose Formulation #3, where their maybe an apparent outlier for subject numbers 1 and 5. If the two subjects are removed the t_{1/2} is 1.5 hours, the same as FORSTEO. The apparent difference in elimination rates may reflect slower wash-in for the subcutaneous product and formulation Formulation #6 when compared with Formulation #3.

Both nasal formulations have very similar t_{1/2} to FORSTEO. Formulation #3 also showed good dose linearity from 200 to 400 μg dose based on the clearance rate and regression analysis. In addition, the formulation was less variable than Formulation #6 and Forsteo based on % coefficient of variation. Accordingly, the intranasal formulations of the invention exceed the Cmax and AUC values for the currently marketed subcutaneous product. This demonstrates that the levels of the marketed product can be exceeded by a nasally administered product, and also that the concentrations of PTH in nasal formulations can be decreased if it is desired to more closely approximate the plasma concentrations of the currently approved product.

### Example 4

### Droplet Size and Spray Characterization

The droplet size and spray characterization of two teriparatide intranasal formulations were evaluated using the Pfeiffer 0.1 ml Nasal Spray Pump 65550 with 36 mm dip tube. The droplet size distribution is characterized by laser diffraction using a Malvern MasterSizer S modular particle size analyzer and a MightyRunt automated actuation station. Single spray droplet distribution is volume weighted measurement. The Spray Pattern is characterized using a SprayVIEW NSP High Speed Optical Spray Characterization System and SprayVIEW NSx Automated Actuation System. The data are shown in Table 17. The diameter of droplet for which 50% of the total liquid volume of sample consists of droplets of 30 micron and 294 micron for formulation #5 and #2, respectively. There are 3% and 1% of the total liquid volume for formulation #5 and #2, respectively, where the droplet size is less than 10 micron. The ellipticity ratio is 1.3 and 1.4 for formulation #5 and #2, respectively.

TABLE 17
Droplet Size and Ellipticity Ratio for Teriparatide Intranasal Formulations

| | | | | % < 10 | Ellip- |
|---|---|---|---|---|---|
| | | | | microm- | ticity |
| | D(v, 0.1) | D(v, 0.5) | D(v, 0.9) | eter | Ratio |
| Formulation #5 | 14 | 30 | 65 | 3 | 1.3 |
| Formulation #2 | 25 | 294 | 676 | 1 | 1.4 |

Although the foregoing invention has been described in detail by way of example for purposes of clarity of understanding, it is apparent to the artisan that certain changes and modifications are comprehended by the disclosure and may be practiced without undue experimentation within the scope of the appended claims, which are presented by way of illustration, not limitation.

What is claimed is:

1. A dosage form of parathyroid hormone (1-34) (PTH) comprising an aqueous pharmaceutical formulation for aerosolized intranasal delivery of PTH having a bioavailability of about 5% or greater, wherein the formulation comprises a therapeutically effective amount of PTH and polysorbate, and wherein least 90% of the PTH can be recovered after storage for 24 weeks at 5° C.

2. The PTH dosage form of claim 1, having greater than about 90% recovery of the PTH after at least six months at 5° C. storage.

3. The PTH dosage form of claim 1, having greater than about 90% recovery of the PTH after one year at 5° C. storage.

4. The PTH dosage form of claim 1, having greater than about 90% recovery of the PTH after two years at 5° C. storage.

5. The PTH dosage form of claim 1, having greater than about 80% recovery of the PTH after 24 weeks at 25° C. storage.

6. The PTH dosage form of claim 1, having greater than about 80% recovery of the PTH after at least six months at 25° C. storage.

7. The PTH dosage form of claim 1, having greater than about 80% recovery of the PTH after one year at 25° C. storage.

8. The PTH dosage form of claim 1, having greater than about 80% recovery of the PTH after two years at 25° C. storage.

9. The PTH dosage form of claim 1, having greater than about 65% recovery of the PTH can be recovered after storage for at least 4 weeks at 40° C.

10. The PTH dosage form of claim 1, having greater than about 90% recovery of the PTH after being in use for greater than about five days.

11. The PTH dosage form of claim 10, having greater than about 90% recovery of PTH at 30° C./65% relative humidity between all sprays.

12. The PTH dosage form of claim 1, wherein the pH is about 5.0 or less.

13. The PTH dosage form of claim 1, wherein the pH is about 4.5 or less.

14. The PTH dosage form of claim 1, wherein the pH is about 4.0 or less.

15. The PTH dosage form of claim 1, wherein the pH is about 3.5 or less.

16. The PTH dosage form of claim 1, wherein the concentration of PTH is at least about 1 mg/ml or greater.

17. The PTH dosage form of claim 1, wherein the concentration of PTH is at least about 2 mg/ml or greater.

18. The PTH dosage form of claim 1, wherein the concentration of PTH is at least about 6 mg/ml or greater.

19. The PTH dosage form of claim 1, wherein the concentration of PTH is at least about 10 mg/ml or greater.

20. The PTH dosage form of claim 1, wherein said dosage form is suitable for intra-nasal administration to achieve a dose of from about 2 μg to about 1000 μg of said PTH.

21. The PTH dosage form of claim 1, wherein said dosage form is suitable for intra-nasal administration to achieve a dose of from about 100 μg to about 600 μg of said PTH.

22. The PTH dosage form of claim 1, wherein polysorbate is present at least about 1 mg/mL in the formulation.

23. The PTH dosage form of claim 1, wherein polysorbate is present at least about 10 mg/mL in the formulation.

24. The PTH dosage form of claim 1, wherein polysorbate is present at least about 50 mg/mL in the formulation.

25. The PTH dosage form of claim 1, further comprising a preservative.

26. The PTH dosage form of claim 25, wherein the preservative is chlorobutanol.

27. A dosage form of parathyroid hormone (1-34) (PTH) comprising an aqueous pharmaceutical formulation for aerosolized intranasal delivery of PTH having a bioavailability of about 10% or greater, wherein the formulation comprises a therapeutically effective amount of PTH, methyl-β-cyclodextrin, didecanoylphosphatidyl choline, and ethylenediaminetetraacetic acid, and wherein least 90% of the PTH can be recovered after storage for 24 weeks at 5° C.

28. The PTH dosage form of claim 27, having greater than about 90% recovery of PTH after at least six months at 5° C. storage.

29. The PTH dosage form of claim 27, having greater than about 90% recovery of PTH after one year at 5° C. storage.

30. The PTH dosage form of claim 27, having greater than about 90% recovery of PTH after two years at 5° C. storage.

31. The PTH dosage form of claim 27, having greater than about 80% recovery of PTH after 24 weeks at 25° C. storage.

32. The PTH dosage form of claim 27, having greater than about 80% recovery of PTH after at least six months at 25° C. storage.

33. The PTH dosage form of claim 27, having greater than about 80% recovery of PTH after one year at 25° C. storage.

34. The PTH dosage form of claim 27, having greater than about 80% recovery of PTH after two years at 25° C. storage.

35. The PTH dosage form of claim 27, having greater than about 65% recovery of the PTH after storage for at least 4 weeks at 40° C.

36. The PTH dosage form of claim 27, having greater than about 90% recovery of the PTH after being in use for greater than about five days.

37. The PTH dosage form of claim 36, having greater than about 90% recovery of PTH at 30° C./65% relative humidity between all sprays.

38. The PTH dosage form of claim 27, wherein the pH is about 5.0 or less.

39. The PTH dosage form of claim 27, wherein the pH is about 4.5 or less.

40. The PTH dosage form of claim 27, wherein the pH is about 4.0 or less.

41. The PTH dosage form of claim 27, wherein the pH is about 3.5 or less.

42. The PTH dosage form of claim 27, wherein the concentration of PTH is at least about 1 mg/ml or greater.

43. The PTH dosage form of claim 27, wherein the concentration of PTH is at least about 2 mg/ml or greater.

44. The PTH dosage form of claim 27, wherein the concentration of PTH is at least about 6 mg/ml or greater.

45. The PTH dosage form of claim 27, wherein the concentration of PTH is at least about 10 mg/ml or greater.

46. The PTH dosage form of claim 27, wherein said dosage form is suitable for intra-nasal administration to achieve a dose of from about 2 μg to about 1000 μg of said PTH.

47. The PTH dosage form of claim 27, wherein said dosage form is suitable for intra-nasal administration to achieve a dose of from about 100 μg to about 600 μg of said PTH.

48. The PTH dosage form of claim 27, further comprising a preservative.

49. The PTH dosage form of claim 48, wherein the preservative is chlorobutanol.
